(19) United States
(12) Patent Application Publication
(10) Pub. No.: US 2008/0226719 A1
(43) Pub. Date: Sep. 18, 2008

(54) Combination of Rosiglitazone and Donepezil for Improvement of Cognitive Function

(76) Inventors: Allen D. Roses, Durham, NC (US); Joanne Heafield, Essex (GB); Ann M. Saunders, Durham, NC (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/067,382

(22) PCT Filed: Sep. 20, 2006
(86) PCT No.: PCT/US06/36597

§ 371 Date: Mar. 19, 2008

Related U.S. Application Data
(60) Provisional application No. 60/719,353, filed on Sep. 22, 2005.
(60) Provisional application No. 60/727,377, filed on Oct. 17, 2005.

(51) Int. Cl. A61K 9/30; A61K 31/4436; A61K 31/4353; G01N 33/53; A61P 25/28

(52) U.S. Cl. 424/475; 514/342; 514/319; 435/7.1

(57) ABSTRACT

Disclosed is a pharmaceutical composition comprising both rosiglitazone or a pharmaceutically acceptable salt thereof and donepezil or a pharmaceutically acceptable salt thereof which is of use in the treatment or prophylaxis of Alzheimer's disease or other dementias and mild cognitive impairment. Also disclosed is an oral dosage forms comprising rosiglitazone or a pharmaceutically acceptable salt thereof and donepezil or a pharmaceutically acceptable salt thereof.

The present invention relates inter alia to a composition, its use in the treatment or prevention of mild cognitive impairment, Alzheimer's disease or other dementias and to methods of treatment or prevention using the composition. In particular, the invention relates to a composition comprising a cholinesterase inhibitor, especially donepezil together with a PPAR-γ agonist, especially rosiglitazone.

Alzheimer's disease (AD) was first described in 1907 by the Bavarian psychiatrist Alois Alzheimer. It is a progressive, debilitating disease and is the most common cause of dementia. Typical symptoms include memory impairment, disordered cognitive function, behavioural changes (including paranoia, delusions, loss of inhibitions) and decline in language function. Pathologically, AD has been traditionally characterised by the presence of two distinct types of brain lesion-neuritic plaques (sometimes referred to as senile plaques) and neurofibrillary tangles.

Neuritic plaques are extracellular amyloid β-protein (Aβ) deposits, typically in a filamentous form, which are around 10 to 150 μm in cross-section and are associated with axonal and dendritic injury. Aβ is formed by the cleavage of amyloid precursor protein (APP) by a series of secretases. Aβ40, a forty residue peptide, is the form of Aβ normally produced in greatest abundance by cells, however, much of the Aβ found within neuritic plaques contains 42 amino acids (Aβ42). Aβ42 is significantly more hydrophobic than Aβ40, and is therefore more prone to aggregation, although Aβ40 is also localised with the plaques. Neuritic plaques are believed to develop over a substantial period of time (months to years). Amyloid depositions in the form of plaques are known to occur prior to the appearance of clinical symptoms, though the correlation between the extent of amyloid deposition and cognitive impairment remains a point of contention.

Neurofibrillary tangles are usually found within the perinuclear cytoplasm of neurons from AD sufferers. The tangles are formed from pairs of filaments which are wound into helices. These highly insoluble filaments have been shown to be composed of the microtubule-associated protein tau in an abnormally hyperphosphorylated state. There is some evidence that the formation of tangles is a response by neurons to the gradual accumulation of Aβ.

Clinically typical AD can be inherited in an autosomal dominant manner however, most cases of the disease (approximately 90%) are considered to be sporadic. These two forms of the disease are phenotypically highly similar save that the rarer familial AD generally presents much earlier than sporadic AD (as such, often known as late-onset AD or LOAD). This general phenotypic similarity suggests that information characterising the mechanism underlying autosomal dominant forms (such as mutations in APP and the presenilin 1 and 2 genes) has relevance to the late-onset sporadic form of AD. Generally, familial AD is associated with increased production of Aβ, whereas sporadic AD may be the result of defective clearance of regular Aβ production.

A large number of contributory factors have been identified for sporadic AD, including: age, low cholesterol concentration, high systolic blood pressure, high glucose concentrations, high insulin concentrations, abnormal glucose tolerance and the presence of an e4 allele of Apolipoprotein E (Kuusisto J et al. BMJ 1997 315:1045-1049).

For further information on AD in general see: Selkoe D Physiol. Rev. 2001 81(2):741-766; Watson G et al. CNS Drugs 2003 17(1):27-45.

Mild cognitive impairment is a condition in which subjects have a slight impairment in cognitive function that is detectable from their pre-morbid baseline, but which also is not sufficiently severe to fulfil diagnostic criteria for AD. As such, MCI may be considered as a transition state between normal cognitive function in a normal aging subject, and the abnormal cognitive function in dementia. MCI can be subdivided into categories based upon the types of cognitive deficits that are detected. A deficit of memory alone typifies amnestic MCI; whereas other types of MCI involve deficits in multiple cognitive domains including memory, or deficits in a single, non-memory domain. The rate of progression from amnestic MCI to AD has been measured in cohort studies to range from 10-20% per year (for more information see Petersen et al. Arch Neurol 2001 58: 1985-1992).

Other dementias which similarly give rise to cognitive deficits include vascular dementia, Lewy body dementia, frontotemporal dementia and dementia associated with Parkinson's disease.

Apolipoproteins are glycoproteins which have been associated with brain development, synaptogenesis and response to neuronal injury. Apolipoprotein E (ApoE) is one protein component of plasma lipoproteins. There are three major isoforms of ApoE (i.e. ApoE2, ApoE3 and ApoE4), which are products of three alleles at a single gene locus. Individuals may therefore be homozygous (APOE2/2, APOE3/3 or APOE4/4) or heterozygous (APOE2/3, APOE2/4 or APOE3/4). The most common allele is APOE3, having an allele frequency in the Caucasian population of approximately 0.78 (Bales K R et al. Mol. Interventions 2002 2: 363-375), and the most common genotype is APOE3/3.

The amino acid sequence of the three isoforms show only slight variation, which is summarized in the Table 1 below.

TABLE 1
Amino-acid sequence variation in apolipoprotein isoforms.

| | ApoE2 | ApoE3 | ApoE4 |
|---|---|---|---|
| Residue 112 | Cysteine | Cysteine | Arginine |
| Residue 158 | Cysteine | Arginine | Arginine |

An association between carriage of an APOE4 allele and the risk of developing AD has been known for some time and is well documented in the literature (Strittmatter W J et al. PNAS 1993 90: 1977-1981; Roses A D Ann Rev Med 1996 47: 387-400). However, APOE genotyping alone is not a sufficient diagnostic test for AD since the presence of the e4 allele is a susceptibility factor and does not cause the disease (Mayeux R et al. New Engl. J. Med. 1998 338:506-511).

The age-adjusted risk of AD in individuals having two APOE4 alleles has been shown to be over three times that of individuals having only one APOE4 allele, which is in turn almost three times that of individuals who do not have an APOE4 allele (Corder et al. Science 1993 261(5123):921-3; Kuusisto J et al. BMJ 1994 309:636-638). Relative to other AD patients, those which are homozygous for APOE4 show an earlier age of onset, increased amyloid burden and decreased acetylcholine levels. The APOE4 allele frequency varies across ethnic populations and has been found to be approximately 0.15 in the Caucasian population but up to 0.4 in patients with AD (Saunders et al. Neurology 1993 43(8): 1467-72).

APOE2, the rarest of the three common alleles, has been suggested to have a protective effect relative to the most common APOE3 allele, individuals having an APOE2 allele generally showing a later onset of disease than those without (Corder et al. Nature Genetics 1994 7(2):180-4; Bales K R et al. Mol Interventions 2002 2: 363-375). The APOE2 allele frequency has been found to be approximately 0.07 in the Caucasian population. There are more recent data that APOE4 status has no bearing on rate of progression once symptoms of possible AD are present.

Glucose metabolism is of critical importance in the function of cells within the central nervous system. Decreases in cerebral glucose metabolism that are regionally specific have been demonstrated in patients with AD (Reiman E M et al. New Eng J Med 1996 334: 752-758; Alexander, G E et al. Am J Psychiatry 2002 159:738-745), both in LOAD and in familial AD (Small G W et al., PNAS 2000 97: 6037-6042).

The decrease in cerebral glucose metabolism in patients at risk for AD has been linked to APOE status, because the regionally specific pattern of decreased cerebral glucose metabolism can be detected many years before the predicted age of onset of clinical symptoms, in individuals who carry one or two APOE4 alleles (Reiman E M et al. New Eng J Med 1996 334: 752-758; Rossor M et al., Annals N Y Acad Sci 1996 772:49-56; Small G W et al., PNAS 2000 97: 6037-6042).

Insulin is also of critical importance in peripheral and central energy metabolism. Secreted by pancreatic β-cells, plasma insulin serves to regulate glucose levels in the blood through periods of feeding and fasting, the rate of glucose uptake in insulin sensitive tissues being controlled by insulin-sensitive glucose transporters. Increases in blood glucose result in the release of insulin, while decreases in blood glucose results in the release of counter-regulatory hormones which increase glucose output by the liver. Type II diabetes results from a reduced ability of insulin to stimulate glucose uptake and to inhibit hepatic glucose output (known as insulin resistance) and an insufficient insulin secretory response to compensate for the insulin resistance.

Insulin is transported across the blood/brain barrier by an insulin receptor-mediated transport process. Peripheral levels of insulin tend to correlate with levels in the central nervous system (CNS), i.e. increased peripheral insulin results in increased CNS insulin. Evidence suggests that insulin has some involvement in normal memory function, and that disorders in peripheral insulin metabolism, such as insulin resistance and hyperinsulinaemia, may have a negative influence on memory. Insulin-promoted increases in glucose utilisation may lead to glycolytic production of acetyl-CoA, the key substrate in the synthesis of the neurotransmitter acetylcholine. Reduction in acetylcholine levels is a key feature of AD.

Peroxisome Proliferator-Activated Receptor gamma (PPAR-gamma) is an orphan member of the steroid/thyroid/retinoid receptor superfamily of ligand-activated transcription factors. PPAR-gamma is one of a subfamily of closely related PPARs encoded by independent genes (Dreyer C et. Al. Cell 1992 68:879-887; Schmidt A et al. Mol. Endocrinol. 1992 6:1634-1641; Zhu et al. J. Biol. Chem. 1993 268:26817-26820; Kliewer S A et al. Proc. Nat. Acad. Sci. USA 1994 91:7355-7359). Three mammalian PPARs have been isolated and termed PPAR-alpha, PPAR-gamma, and PPAR-delta (also known as NUC-1). These PPARs regulate expression of target genes by binding to DNA sequence elements, termed PPAR response elements (PPRE). To date, PPREs have been identified as the enhancers of a number of genes encoding proteins that regulate lipid metabolism, suggesting that PPARs play a pivotal role in the adipogenic signalling cascade and lipid homeostasis (Keller H et al. Trends Endocrin. Met. 1993 4:291-296).

European Patent 306228 describes a class of PPAR-gamma agonists which are thiazolidinedione derivatives for use as insulin sensitizers in the treatment of Type II diabetes mellitus. These compounds have anti-hyperglycaemic activity. One preferred compound described therein is known by the chemical name 5-[4-[2-(N-methyl-N-(2-pyridyl)amino)ethoxy]benzyl]thiazolidine-2,4-dione and has been given the generic name rosiglitazone. Salts of this compound, including the maleate salt, are described in WO94/05659. European Patent Applications, Publication Numbers: 0008203, 0139421, 0032128, 0428312, 0489663, 0155845, 0257781, 0208420, 0177353, 0319189, 0332331, 0332332, 0528734, 0508740; International Patent Applications, Publication Numbers 92/18501, 93/02079, 93/22445 and U.S. Pat. Nos. 5,104,888 and 5,478,852, also disclose certain thiazolidinedione PPAR-gamma agonists. Specific compounds that may be mentioned include 5-[4-[2-(5-ethyl-2-pyridyl)ethoxy]benzyl]thiazolidine-2,4-dione (also known as pioglitazone), 5-[4-[(1-methylcyclohexyl)methoxy]benzyl]thiazolidine-2,4-dione (also known as ciglitazone), 5-[[4-[(3,4-dihydro-6-hydroxy-2,5,7,8-tetramethyl-2H-1-benzopyran-2-yl)methoxy]phenyl]methyl]-2,4-thiazolidinedione (also known as troglitazone) and 5-[(2-benzyl-2,3-dihydrobenzopyran)-5-ylmethyl)thiazolidine-2,4-dione (also known as englitazone).

U.S. Pat. No. 6,294,580 (the disclosure of which is herein incorporated by reference) describes a series of PPAR gamma agonist compounds not of the thiazolidinedione class but which are instead O— and N— substituted derivatives of tyrosine which nevertheless are effective as insulin sensitizers in the treatment of Type II diabetes mellitus. One such compound has chemical name N-(2-benzoylphenyl)-O—[2-(5-methyl-2-phenyl-4-oxazolyl)ethyl]-L-tyrosine (also known as 2(S)-(2-Benzoyl-phenylamino)-3-{4-[2-5-methyl-2-phenyl-oxazol-4-yl)-ethoxy]-phenyl}-propionic acid, or by the generic name farglitazar).

A body of clinical evidence suggests that impairment of cerebral glucose metabolism is present during AD, and in APOE4-carriers before the clinical onset of symptoms of AD (Reiman E M et al. New Eng J Med 1996 334: 752-758; Rossor M et al., Annals NY Acad Sci 1996 772:49-56; Small G W et al., PNAS 2000 97: 6037-6042).

Converging clinical and epidemiological evidence also suggests that the risk of developing AD may be influenced by insulin resistance. However, the exact nature of the relationship between insulin resistance and AD is complex and not presently fully understood.

Hyperinsulinaemia has been shown to be a risk factor for AD. In one study it was concluded by the authors to be independent of APOE genotype (Kuusisto J et al. BMJ 1997 315:1045-1049), where hyperinsulinaemic elderly subjects without an APOE4 allele (APOE4−) had an AD prevalence of 7.5% in hyperinsulinaemic subjects, compared with 1.4% in normoinsulinaemic subjects; while hyperinsulinaemic elderly subjects with an APOE4 allele (APOE4+) had an AD prevalence of 7.0% hyperinsulinaemic subjects, compared with 7.1% in normoinsulinaemic subjects. Other studies have indicated a link between APOE genotype and insulin resistance (Watson G et al. CNS Drugs 2003 17(1):27-45).

For example, patients who were not homozygous for the APOE4 allele have abnormalities of insulin metabolism (specifically increased plasma insulin levels), suggesting a possible factor in the development of AD in these patients, while those who were homozygous for the APOE4 allele demonstrated normal peripheral levels of insulin. Both groups demonstrated reduced cerebrospinal fluid insulin levels compared to non-AD subjects (Craft S et al. Neurology 1998 50:164-168). Furthermore, patients without an APOE4 allele have reduced rates of insulin-mediated glucose disposal relative to those who are APOE4+ (Craft S et al. Neuroendocrinology 1999 70:146-152).

It is well accepted that normal cholinergic signalling is a necessity for the proper function of mental processes such as memory. A large body of evidence indicates that AD patients have abnormalities in cholinergic signalling, the extent of which correlates with the level of cognitive impairment. As with many aspects of AD research the link between the progression of the disease and the observed cholinergic dysfunction is not fully understood. To date the use of agonists for muscarinic or nicotinic acetylcholine receptors has not proved to be of clinical value, though a number of cholinesterase inhibitors have demonstrated sufficient efficacy with an acceptable degree of adverse effects to be approved for use in the treatment of AD, these include tacrine (Cognex™), galantamine (Reminyl/Radazyne™), rivastigamine (Exelon™) and donepezil (Aricept™). For further information see, for example, Terry A V et al. J. Pharmacol. Exp. Ther. 2003 306(3):821-827.

In a recently published study investigating the use of donepezil in individuals with mild cognitive impairment (a transitional state between normal aging and early AD), donepezil was shown to reduce the rate at which patients developed AD during the first twelve months of administration, although at three years there was no separation between groups. Additionally, the beneficial effect seen in the first 12 months was then followed by a more acute deterioration in the following 24 months. Although no significant difference in the general intent to treat population was observed after three years compared to placebo, patients who were carriers of one or two copies of an APOE4 allele had a reduced risk of progressing to AD compared to placebo (Petersen R et al. New Engl. J. Med. 2005 352:2379-2387).

The use of insulin sensitizers in the treatment of AD has been proposed previously. International patent application WO98/39967 discloses a method for the treatment or prevention of AD by administering an agent which reduces serum insulin levels, such as a thiazolidinedione. International patent application WO99/25346 discloses a method for the treatment or prevention of a disease mediated by apoptosis, such as neurodegenerative disorders including AD and Parkinson's disease by administering an apoptosis inhibitor, for example an insulin sensitising agent such as rosiglitazone. International patent application WO00/32190 discloses a method for the treatment or prevention of AD by administering a PPAR-gamma agonist, such as the thiazolidinediones pioglitazone and rosiglitazone. International patent application WO00/35437 discloses methods of improving mental performance in subjects suffering from reduced mental performance by the administration of insulin sensitising agents, such as the thiazolidinediones pioglitazone and rosiglitazone.

In Parkinson's disease models, there is evidence that thiazolidinediones (including rosiglitazone and pioglitazone) can protect dopaminergic cells from various toxic insults including acetaldehyde (Jun et al (2006) Biochem Biophys Res Comm 340, 221-227), MPTP (Dehmer et al (2004) J Neurochem 88, 494-501) and 8-OHDA (Chen et al (2004) FASEB 18, 1162-1164).

Although PPAR-γ agonists such as rosiglitazone and cholinesterase inhibitors such as donepezil have both been suggested for use in the treatment of mild cognitive impairment and AD, there is no indication in any of the prior art that they could be used in combination.

Therefore, in a first aspect of the invention there is provided a composition comprising rosiglitazone or a pharmaceutically acceptable salt thereof and donepezil or a pharmaceutically acceptable salt thereof together with a pharmaceutically acceptable diluent or carrier.

It is implicit that the pharmaceutically acceptable diluent or carrier may be a mixture of substances i.e. a combination of one or more pharmaceutically acceptable diluents or carriers.

WO03/061648 relates to the use of an acetylcholinesterase inhibitor such as donepezil for reducing insulin resistance and for treating type II diabetes. The document teaches that other drugs used in the treatment of diabetes may be combined with the acetylcholinesterase inhibitor and these other drugs include insulin and insulin analogues; type II diabetes drugs such as sulfonylureas, biguanides, alpha-glucosidase inhibitors, thiazolidinones, and meglitinides; phosphodiesterase inhibitors; cholinergic agonists; nitric oxide donors; antioxidants; and glutathione increasing compounds. Although rosiglitazone is one of the drugs identified in the long list, there is no suggestion that a combination of an acetylcholinesterase inhibitor with rosiglitazone would be any more favourable than a combination with one of the numerous other drugs on the lists provided. There are no examples of any acetylcholinesterase inhibitor in combination with another drug.

WO2005/074917 relates to a method of treating diabetes mellitus or conditions associated with diabetes, such as cognitive impairment using a phenserine-like compound such as donepezil, galantamine or tacrine. The document teaches that the phenserine like compound may be used in combination with a hypoglycaemic agent such as a sulfonyl urea, meglitinide, biguanide, thiazolidinedione or α-glucosidase inhibitor. Once again, there are no examples of a phenserine-like compound in combination with another drug and no suggestion that rosiglitazone would be of any more use than any of the other hypoglycaemic agents suggested.

In contrast to the prior art, the composition of the present invention is intended not for the treatment of diabetes but for improving cognitive function in a subject suffering from or susceptible to mild cognitive impairment, Alzheimer's disease or other dementias.

Therefore, the invention further provides a composition comprising rosiglitazone or a pharmaceutically acceptable salt thereof and donepezil or a pharmaceutically acceptable salt thereof together with a pharmaceutically acceptable diluent or carrier for use in improving cognitive function in a subject suffering from or susceptible to mild cognitive impairment, Alzheimer's disease or other dementias.

Furthermore, there is provided a method for improving cognitive function in a subject suffering from or susceptible to mild cognitive impairment, Alzheimer's disease or other dementias, the method comprising administering to the subject a safe and effective amount of a composition comprising rosiglitazone or a pharmaceutically acceptable salt thereof and donepezil or a pharmaceutically acceptable salt thereof.

In yet another aspect, the invention further provides the use of a composition comprising rosiglitazone or a pharmaceutically acceptable salt thereof and donepezil or a pharmaceutically acceptable salt thereof in the preparation of an agent for improving cognitive function in a subject suffering from or susceptible to mild cognitive impairment, Alzheimer's disease or other dementias.

A further aspect of the invention provides a combination of rosiglitazone or a pharmaceutically acceptable salt thereof and donepezil or a pharmaceutically acceptable salt thereof for simultaneous, separate or sequential use in improving cognitive function in a subject suffering from or susceptible to mild cognitive impairment, Alzheimer's disease or other dementias. A related aspect provides a method of improving cognitive function in a subject suffering from or susceptible to mild cognitive impairment, Alzheimer's disease or other dementias which comprises administering to said subject rosiglitazone or a pharmaceutically acceptable salt thereof and donepezil or a pharmaceutically acceptable salt thereof for simultaneous, separate or sequential use in combination. A related aspect provides use of rosiglitazone or a pharmaceutically acceptable salt thereof and donepezil or a pharmaceutically acceptable salt thereof in the manufacture of a medicament for simultaneous, separate or sequential use in combination for improving cognitive function in a subject suffering from or susceptible to mild cognitive impairment, Alzheimer's disease or other dementias.

There have been conflicting reports concerning the effect of the APOE4 allele on treatment of mild cognitive impairment, Alzheimer's disease or other dementias with cholinesterase inhibitors. Poirier et al (Proc. Natl. Acad. Sci. U.S.A. (1995), 92, 12260-12264) teach that when patients with AD are treated with tacrine, 83% of the non-APOE4 carriers showed an improvement in cognitive function, whereas 60% of the APOE4 carriers were unchanged or worse after 30 weeks.

However, Greenberg et al (Arch. Neurol., (2000), 57, 94-99) state that they could not confirm an association between the APOE4 allele and the lack of response to donepezil therapy and Winblad et al (Neurology (2001), 57, 489-495) teach that APOE genotype did not affect the beneficial response to donepezil treatment.

Prior to the earliest priority date of this patent application, there has been no definitive evidence which shows that the use of PPAR-gamma agonists such as rosiglitazone to improve cognitive function in subjects suffering from or susceptible to MCI, AD or other dementias provides benefit only to those subjects who are not homozygous for the APOE4 allele, and provides most benefit to those who are non-carriers of the APOE4 allele.

However, the inventors have now shown that the PPAR-γ agonist rosiglitazone is significantly more effective for the treatment of patients who are not homozygous for the APOE4 allele and most effective in the treatment of patients who do not carry the APOE4 allele.

Therefore, the composition of the present invention may provide the greatest benefit to patients who are not homozygous for the APOE4 allele and in the aspects of the invention described above it is preferred that the subject has been pre-determined not to be homozygous for the APOE4 allele. The subject may, for example, have been pre-determined to be APOE4−.

According to another aspect of the present invention there is provided a method for improving cognitive function in a subject suffering from or susceptible to MCI, Alzheimer's disease or other dementias, which subject is not homozygous for the APOE4 allele, comprising the steps of:
i) screening the subject to determine that the subject is not homozygous for the APOE4 allele; and then
ii) administering a safe and effective amount of a composition comprising rosiglitazone or a pharmaceutically acceptable salt thereof and donepezil or a pharmaceutically acceptable salt thereof to said subject.

Screening method (i) may involve determining whether the subject has an APOE2 or an APOE3 allele.

In one embodiment of the invention, screening step (i) involves determining that the subject carries a single copy of the APOE4 allele. For example the subject may be determined to be APOE3/APOE4.

In a more preferred embodiment of the invention screening step (i) involves determining that the subject is APOE4− (i.e. does not carry the APOE4 allele). For example the subject may be determined to be APOE3/APOE3 or APOE2/APOE3.

For example the subject may be suffering from or be susceptible to (for example may be suffering from) MCI or AD. In one embodiment of the invention the subject is suffering from MCI (particularly amnestic MCI). In another embodiment of the invention the subject is suffering from Alzheimer's disease. In another embodiment of the invention the subject is susceptible to MCI (particularly amnestic MCI). In another embodiment of the invention the subject is susceptible to Alzheimer's disease. In further embodiments the subject is suffering from or susceptible to other dementias such as vascular dementia, Lewy body dementia, frontotemporal dementia or dementia associated with Parkinson's disease.

Also provided according to the present invention is a method of screening a subject suffering from or susceptible to or susceptible to MCI, Alzheimer's disease or other dementias as an aid in predicting the subject's response to administration of a composition comprising rosiglitazone or a pharmaceutically acceptable salt thereof and donepezil or a pharmaceutically acceptable salt thereof, comprising screening to determine whether the subject carries zero or 1 copy of the APOE4 allele.

The method may in particular include screening to determine whether the subject is APOE4−.

There is also provided a kit comprising (i) a composition comprising rosiglitazone or a pharmaceutically acceptable salt thereof and donepezil or a pharmaceutically acceptable salt thereof and (ii) instructions directing administration of the PPAR gamma agonist (typically in the form of a pharmaceutical composition) to a subject who is not homozygous for the APOE4 allele (for example a subject who has been pre-determined not to be homozygous for the APOE4 allele). For example the instructions direct administration of the composition to a subject suffering from or susceptible to MCI, Alzheimer's disease or other dementias who is not homozygous for the APOE4 allele. According to a particular aspect of the invention, the subject is APOE4− (for example the subject has been pre-determined not to have any copy of the APOE4 allele).

A kit may optionally contain one or more reagents for determining whether a subject has zero, one or two APOE4 alleles. Such reagents may typically be selected from the group consisting of a probe, a primer, an antibody or a combination thereof.

Alternatively in the above aspects of the invention the subject may carry, or may be determined or pre-determined to carry, a single copy of the APOE4 allele. For example the subject may be or may be determined or pre-determined to be APOE3/APOE4.

As shown in the Examples below, the inventors have unexpectedly discovered that the PPAR-γ agonist rosiglitazone produces a clinically relevant improvement in cognitive function relative to placebo in subjects with mild to moderate AD who do not carry the APOE4 allele. The results suggest that patients who carry one copy of the APOE4 allele experience a stabilisation in cognitive function (i.e. neither significant improvement nor decline) on treatment with rosiglitazone. The results suggest that patients who are homozygous for the APOE4 allele may experience a clinical decline on treatment with rosiglitazone, although it is not clear whether or not the decline was a result of treatment or due to natural progression of the disease.

Without being limited by theory, the inventors have attempted to rationalize this invention. According to one theory, the amino acid sequence differences between the isoforms results in a difference in their protein folding. In particular ApoE2 and ApoE3 are characterised by the presence of Cys at position 112 and Arg at position 61. ApoE4 is characterised by the presence of Arg at position 112 and Arg at position 61. Residues 61 and 112 interact in the folded protein and since Arg is positively charged and Cys is negatively charged the ApoE2 and ApoE3 protein folding is tighter in this region than the ApoE4 protein folding. Although all ApoE isoforms experience intracellular degradation, it is believed that as a result of conformational differences between the isoforms, ApoE4 experiences a faster rate of degradation. The fragments produced in degradation have lipid and receptor binding sites that in concert cause mitochondrial toxicity. The lipid binding site of the ApoE4 fragment appears to be a more avid binder of lipids than that of the ApoE2 or ApoE3 fragments. The ApoE4 fragment therefore binds to and disrupts mitochondria to a greater extent than the ApoE2 and ApoE3 fragments; this disruption also affects mitochondrial transport from the soma to the synapse. This disruption can also render mitochondria less responsive to increasing glucose or lactate substrate which is a consequence of treatment with PPAR-γ agonists. The effect would be expected to be greater for subjects with 2 copies of the APOE4 allele than those with one copy and greater for subjects with one copy of the APOE4 allele than those carrying no copies.

The predetermination of whether the subject carries zero, one or two copies of the APOE4 allele may, for example, be carried out by the APOE4 screening methods described herein.

In one embodiment of the invention the subject will suffer from Type II diabetes. In another embodiment of the invention the subject will not suffer from Type II diabetes.

Procedures for the diagnostic screening of subjects to determine the presence or absence of the APOE4 allele are well documented in the literature and are within the capabilities of one skilled in the art.

The absence of the APOE4 allele may be determined directly, by a negative result in tests which indicate the presence of the allele, or indirectly, for example by positive results in tests which indicate the presence of the APOE2 and APOE3 alleles (thereby excluding the possibility that an APOE4 allele is present).

Screening methodology may be based in a number of approaches such as isoelectric focusing methods, immunological methods, immunochemical methods or sequencing methods (either of the ApoE protein itself or of the nucleic acids encoding it). Specific methods include PCR-based methods using restriction fragment enzymes or TaqMan primers.

Immunological methods involve the detection of ApoE isoforms by the use of isoform specific antibodies. However, immunological detection methods may be hampered by problems with antibody cross-reactivity, which can impact the reliability of results.

Immunochemical methods include those described in International Patent Application WO94/09155 (related to granted patents EP0625212, JP03265577 and U.S. Pat. No. 5,508,167), which discloses methods for detecting the presence or absence of ApoE4 for the diagnosis of AD. The methods for detecting the presence or absence of ApoE4 disclosed in WO94/09155 are also of use in the practice of the present invention. Briefly, a sample from the subject (e.g. a blood sample) is contacted with a solid support designed to react specifically with sulfhydryl groups. The liquid sample is then separated from the solid support and tested for the presence of ApoE by the use of an appropriate antibody. The presence of ApoE4 in the separated sample indicates that the subject is a carrier of the APOE4 allele. Unlike ApoE2 and ApoE3, the ApoE4 protein does not contain any cysteine residues and therefore does not react with and become immobilised onto the solid support. The presence of unbound ApoE in the liquid sample after passing over the solid support indicates that the individual is ApoE4+; the absence of ApoE immunoreactivity in the liquid sample after passing over the solid support indicates that the individual is ApoE4−. Issues with antibody specificity are largely negated by this approach, since it does not require the immunological differentiation of ApoE isoforms.

Sequencing approaches involve the isolation and purification of either ApoE protein or the DNA encoding ApoE from the subject, determination of the amino-acid or DNA sequence by conventional means, and comparison of the results with known amino-acid or DNA sequences for the different alleles.

The preferred method of determining APOE genotype involves using PCR-based methods—primarily PCR of a portion of the APOE gene followed by digestion with restriction enzymes that recognize the DNA substitutions that distinguish the alleles and gel electrophoresis or most currently, using TaqMan real time PCR. Specifically, APOE genotyping may be performed using an established Taqman protocol, a fluorescence detection system that relies upon a 5′-nuclease assay with allele specific fluorogenic probes. These probes only fluoresce when they are bound to the template. This method is described in Macleod et al. Eur J Clinical Investigation 2001 31(7):570-3. Commercial products for determining APOE genotype are available from LabCorp and Athena Diagnostics.

Improvement in cognitive function in a patient may be determined by one or more established methods for example ADAS-cog and/or CIBIC+ and/or the DAD method (details of each of which are described elsewhere herein, and the associated references are herein incorporated in their entirety by reference). The preferred method is ADAS-cog. Suitably the improvement in ADAS-cog is at least 1 point, especially at least 2 points over a 24 week treatment period.

Another possible method is the Buschke Selective Reminding Test (Grober E et al. Neurology 1988 38:900-903).

By “improvement in cognitive function” is meant an improvement in cognitive treatment with drug treatment over the passage of time relative to an untreated individual. Since dementia (eg AD) patients typically decline in cognitive function with time an “improvement in cognitive function” embraces a slowing or arrest in decline as well as absolute improvement. As is shown in Example 2, an absolute improvement in cognitive function does appear to result from preferred methods of performing the invention.

It will be clear to those skilled in the art that the rosiglitazone or a pharmaceutically acceptable salt thereof and donepezil or a pharmaceutically acceptable salt thereof may be presented in the form of pharmaceutically acceptable solvates.

Suitable solvates include hydrates.

Suitable pharmaceutically acceptable salts of rosiglitazone and donepezil include those formed with both organic and inorganic acids or bases.

Pharmaceutically acceptable acid addition salts include those formed from hydrochloric, hydrobromic, sulphuric, citric, tartaric, phosphoric, lactic, pyruvic, acetic, trifluoroacetic, triphenylacetic, sulphamic, sulphanilic, succinic, oxalic, fumaric, maleic, malic, glutamic, aspartic, oxaloacetic, methanesulphonic, ethanesulphonic, arylsulphonic (for example p-toluenesulphonic, benzenesulphonic, naphthalenesulphonici or naphthalenedisulphonic), salicylic, glutaric, gluconic, tricarballylic, cinnamic, substituted cinnamic (for example, phenyl, methyl, methoxy or halo substituted cinnamic, including 4-methyl and 4-methoxycinnamic acid), ascorbic, oleic, naphthoic, hydroxynaphthoic (for example 1- or 3-hydroxy-2-naphthoic), naphthaleneacrylic (for example naphthalene-2-acrylic), benzoic, 4 methoxybenzoic, 2- or 4-hydroxybenzoic, 4-chlorobenzoic, 4-phenylbenzoic, benzeneacrylic (for example 1,4-benzenediacrylic) and isethionic acids.

Pharmaceutically acceptable base salts include ammonium salts, alkali metal salts such as those of sodium and potassium, alkaline earth metal salts such as those of calcium and magnesium and salts with organic bases such as dicyclohexylamine and N-methyl-D-glucamine.

It is particularly preferred that, independently or in combination, the rosiglitazone is in the form of rosiglitazone maleate and the donepezil is in the form of donepezil hydrochloride.

Donepezil and its salts may also exist in several polymorphic forms and any of these forms may be used in the present invention.

Suitable formulations include those for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), transdermal (eg via skin patch), rectal and topical (including dermal, buccal, sublingual and intraocular) administration, although the most suitable route may depend upon for example the condition of the recipient. The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of use in the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-lowing form such as a powder or granules, optionally mixed with a binder, lubricant, inert diluent, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release or sustained release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di- or poly-saccharides (e.g. lactose or starch). Use of lactose is preferred.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants e.g. oleic acid or lecithin and cosolvents e.g. ethanol. Pressurised formulations will generally be retained in a canister (e.g. an aluminium canister) closed with a valve (e.g. a metering valve) and fitted into an actuator provided with a mouthpiece.

Medicaments for administration by inhalation desirably have a controlled particle size. The optimum particle size for inhalation into the bronchial system is usually 1-10 um, preferably 2-5 um. Particles having a size above 20 um are generally too large when inhaled to reach the small airways. To achieve these particle sizes the particles of the active ingredient as produced may be size reduced by conventional means e.g. by micronisation. The desired fraction may be separated out by air classification or sieving. Preferably, the particles will be crystalline. When a diluent/excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 um and not less than 15% will have a MMD of less than 15 um.

Intranasal sprays may be formulated with aqueous or non-aqueous vehicles with the addition of agents such as thickening agents, buffer salts or acid or alkali to adjust the Ph, isotonicity adjusting agents or anti-oxidants.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as glycerin and glycerine or sucrose and acacia.

It should be understood that in addition to the ingredients particularly mentioned above, the formulations of this invention may include other agents conventional in the art having regard to the type of formulation in question, for example those suitable for oral administration may include flavouring agents.

It is preferred that the composition is formulated for oral administration.

One complication which occurs when formulating a composition comprising both rosiglitazone and donepezil, is that the two drugs have widely different pharmacokinetic characteristics. To obtain the optimum pharmacokinetic profile of rosiglitazone, the drug formulated into an immediate release dosage form is sometimes administered to a subject twice per day. If a modified dosage form is provided such as that described in WO 2005/013935, the drug need only be administered once per day. This document teaches formulations of rosiglitazone which comprise a core which contains two different active compositions, an immediate release formulation and a modified release formulation. The tablet is surrounded by a coating, for example ethyl cellulose, through which holes penetrate, one to the immediate release depot and one to the modified release depot. The arrangement ensures a highly controlled release of rosiglitazone which obtains the optimal pharmacokinetic profile.

Donepezil, on the other hand has different pharmacokinetic characteristics to rosiglitazone and in general it is not necessary to provide a modified release formulation in order to achieve a once a day dosage regime.

This means that any oral formulation containing both rosiglitazone and donepezil should be designed to ensure that acceptable pharmacokinetic profiles are achieved for both drugs, particularly if a once-a-day dosage form is required.

Surprisingly, the present inventors believe that it is possible to produce a similar formulation to that of WO 2005/013935 in which donepezil is included in the immediate release formulation, without substantially affecting the release profile of the rosiglitazone. Such a formulation forms a further aspect of the present invention.

Therefore, in yet another aspect of the present invention, there is provided an oral dosage form comprising:
a first composition comprising rosiglitazone or a pharmaceutically acceptable salt thereof and donepezil or a pharmaceutically acceptable salt thereof formulated for immediate release on contact with aqueous media; and
a second composition comprising rosiglitazone or a pharmaceutically acceptable salt thereof formulated for modified release on contact with aqueous media.

The term “modified release” means a composition which has been designed to produce a desired pharmacokinetic profile and includes delayed pulsed and sustained release systems, either alone or in any combination. In general, however, the second composition is formulated for sustained release, optionally combined with delayed release.

The oral dosage form may, for example, take the form of a tablet in which the first and second compositions are arranged in two or more separate layers, preferably two layers.

Alternatively, the first and second compositions may be multiparticulates.

Multi-particulates include drug-coated or drug containing non-pareil substrates, for example lactose spheres.

When the oral dosage form comprises multiparticulates, those of the first composition will generally be uncoated, or have a non-functional coat, whereas those of the second composition will usually have a functional coat such as an enteric coat. In this embodiment, the first composition may comprise beads which contain or are coated with both rosiglitazone or a pharmaceutically acceptable salt thereof and donepezil or a pharmaceutically acceptable salt thereof or, alternatively comprise a mixture of beads containing or coated with rosiglitazone or a pharmaceutically acceptable salt thereof and beads containing or coated with donepezil or a pharmaceutically acceptable salt thereof. The multiparticulates of this embodiment may be filled into a capsule.

When the immediate release composition is in tablet form, suitable diluents include saccharoses, for example lactose and maltose. More suitably the composition is predominantly lactose, with the optional addition of other formulation aids such as magnesium stearate.

Alternatively, as discussed above, the immediate release composition may be in the form of multiparticles which are uncoated to allow immediate release of the rosiglitazone or a pharmaceutically acceptable salt thereof and donepezil or a pharmaceutically acceptable salt thereof.

Delayed release is conveniently obtained by use of a gastric resistant formulation such as an enteric formulation, which may comprise multi-particulates, such as multi-particulate spheres, coated with a gastric resistant polymer. Suitable gastric resistant polymers are well known in the art and are listed, for example, in WO 2005/013935. They include polymers such as methacrylates, cellulose acetates, carbomers, polyvinyl acetate phthalate and hydroxypropyl methylcellulose phthalate.

Sustained release provides release of the active agent over a time period of up to 26 hours, suitably 4 to 24 hours, more preferably 12 to 24 hours.

Sustained release is typically provided by use of a sustained release matrix, usually in tablet form, non-disintegrating or eroding matrices.

The sustained release matrix may be a tablet formulation. Suitable non-disintegrating matrix tablet formulations are known in the art and are provided, for example by the incorporation of one or more of methacrylates, cellulose acetates, carbomers and hydroxypropylmethyl cellulose into the tablet.

Alternatively, sustained release can be achieved by the provision of multiparticles (as defined above) coated with semipermeable membranes such as ethylcellulose polymer.

The oral dosage form described above may be coated with an enteric coat and therefore, in yet another aspect of the present invention, there is provided an oral dosage form comprising an erodable core and an erodable coating around the core, wherein the core comprises:
a first composition comprising rosiglitazone or a pharmaceutically acceptable salt thereof and donepezil or a pharmaceutically acceptable salt thereof formulated for immediate release on contact with aqueous media; and
a second composition comprising rosiglitazone or a pharmaceutically acceptable salt thereof formulated for sustained release on contact with aqueous media.

The erodable coating may further comprise one or more openings extending substantially completely through said coating but not penetrating the core and communicating from the environment of use to the core, wherein release of the rosiglitazone and donepezil from the core occurs substantially through said opening(s) and through erosion of the coating under pre-determined pH conditions.

The core may comprise a single layer of each of the first and second compositions.

It is preferred that the erosion of the coating is pH dependent and may disintegrate partially or wholly, become porous or dissolve on contact with an environmental fluid to allow the fluid to contact the core. Preferably, the erodable coating is an enteric coating, i.e. it has a defined, pre-determined pH threshold at which it dissolves and which is preferably greater than pH 4.5, more preferable pH 4.5 to 8 and most preferably in the range pH 5 to 7. It is also preferred that the enteric coating is non permeable.

Materials and their blends suitable for use as a pH-dependent erodable coating material are well known and are described, for example in WO 2005/013935. Particularly suitable enteric coating materials include those based on methacrylic acid polymers and copolymers such as that sold under the trade mark Eudragit L30.

Typical size openings in the coat are again as described in WO 2005/013935. In general they are in the range of about 0.19 to about 50.3 mm², which corresponds to a circular opening having a diameter in the range of 0.5 mm to 8 mm, preferably 2 mm to 4 mm. The size of the opening(s) will depend on the overall size of the tablet and the desired rate of release. The opening(s) may have any shape but a rounded shape, for example substantially circular or elliptical is generally preferred.

The openings may be formed by methods described in WO 2005/013935, for example by drilling using mechanical bits or laser beams or by punches. Alternatively, the opening(s) may be formed in situ by forming a coating containing pore-forming agents, i.e. material that will dissolve in the stomach to create pores in the coating.

The opening(s) may comprise 0.25 to 70%, more typically 10-70% of the total face area.

The coating may be provided with one, two or more openings and an embodiment with two openings is particularly suitable when the core comprises discrete layers of the first and second compositions. In this case the core and coating may be arranged such that a first opening provides access to the first composition and a second opening provides access to the second composition.

As a protection for the core material, to prevent contamination via the openings before dosing, it may be desirable to provide a conventional seal coating to either the core or the dosage form after formation of the opening(s).

The dosage forms described above should allow the rosiglitazone and donepezil to be released in such a way that the plasma concentration of both agents is maintained at an optimum level over a 24 hour period so that a combined once-a-day dosage form becomes a possibility.

As already described above, the inventors have shown that rosiglitazone is most effective in the treatment of subjects who are not homozygous for APOE4, and in particular subjects who do not carry the APOE4 allele.

Therefore, the uses and methods described above for combinations of rosiglitazone and donepezil apply equally to the specific oral dosage forms of this aspect of the invention.

Although it is preferred that the rosiglitazone and donepezil are co-formulated, alternatively that they may be administered in separate compositions simultaneously or sequentially. Typically when the rosiglitazone and donepezil are not administered at the same time they are administered within such time that they have therapeutically effective plasma levels for an overlapping period. Suitably when rosiglitazone is administered in a composition not containing donepezil it is formulated for once per day administration. When each active is formulated for once-per-day administration, it would be sufficient that each active is taken daily.

A suitable daily dosage for rosiglitazone (eg as rosiglitazone maleate) will typically be in the range 0.01 mg to 12 mg (for example 2 mg, 4 mg or 8 mg daily). A daily dose of 8 mg or more eg 8 mg may be especially suitable. In the context of the application of the present invention to APOE4 heterozygotes, administration of higher doses of rosiglitazone (eg 4 mg or more, for example 4 mg or 8 mg) would seem to be advantageous.

A suitable daily dosage for donepezil (eg as donepezil hydrochloride) will typically be in the range 2-15 mg, eg 5 mg or 10 mg.

Thus suitable dosage forms according to the present invention may contain 2 mg, 4 mg or 8 mg rosiglitazone (eg as rosiglitazone maleate) and 5 mg or 10 mg donepezil (eg as donepezil hydrochloride).

It will be understood that the combination of medicaments of the invention may find use in prophylaxis as well as (more suitably) in the treatment of subjects suffering from mild cognitive impairment, Alzheimer's disease or other dementias.

The invention will now be further described with reference to the examples and to the drawings in which:

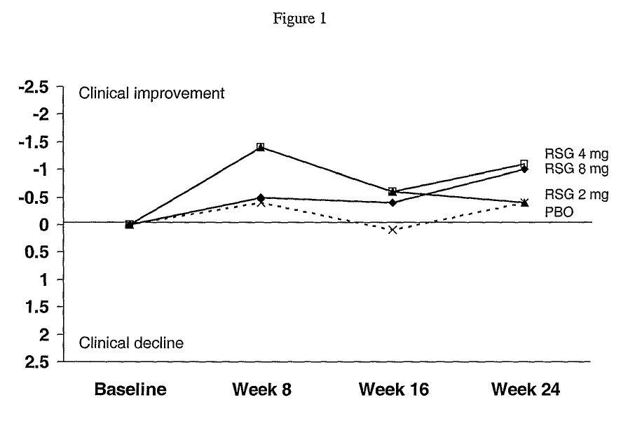

FIG. 1 shows the model adjusted ADAS-cog change from baseline in the intent to treat population of Example 2.

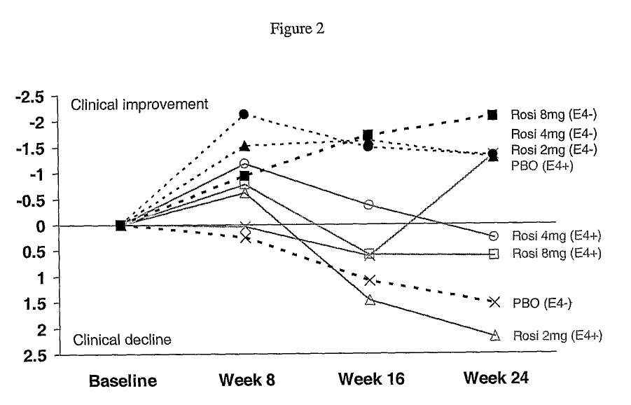

FIG. 2 shows the model adjusted ADAS-cog change from baseline in the genotyped population of Example 2 by treatment regime and APOE allele status.

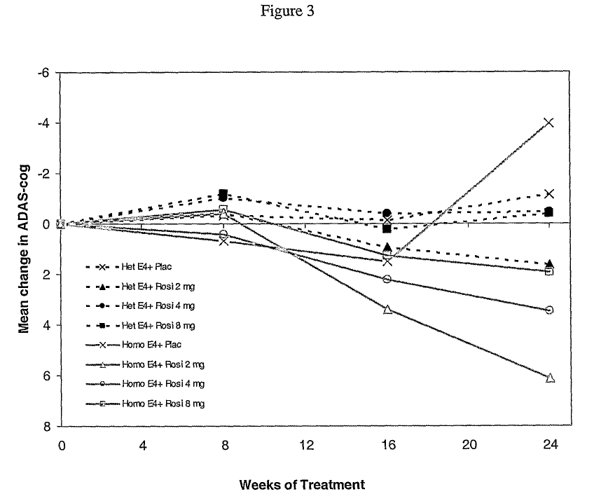

FIG. 3 shows a plot of model adjusted ADAS-cog change from baseline in the APOE4 heterozygote (“Het”) and APOE4 homozygote (“Homo”) populations of Example 2.

### EXAMPLES

Examples 1 and 2 relate to a formulation containing rosiglitazone maleate and demonstrate that treatment with rosiglitazone is most effective in patients without the APOE4 allele and has the least therapeutic effect in patients who are homozygous for APOE4.

Example 3 relates to a variety of formulations containing rosiglitazone in combination with donepezil.

### Example 1

### Preparation of Rosiglitazone Maleate Extended Release Tablets

Extended release tablets containing 2 mg, 4 mg or 8 mg of the PPAR-gamma agonist rosiglitazone (in the form of the maleate salt) were prepared according to the methods described in WO05/013935 (corresponding to Example 3 therein).

### (a) 2 mg Rosiglitazone Extended Release Tablet

A core was formed from the following compositions:

TABLE 2
2 mg rosiglitazone tablet first composition (immediate release layer).

| Component | Proportion (% w/w) |
|---|---|
| Rosiglitazone (as maleate salt) | 1.99 |
| Lactose | 97.48 |
| Yellow iron oxide | 0.03 |
| Magnesium stearate | 0.5 |

TABLE 3
2 mg rosiglitazone tablet second composition (modified release layer).

| Component | Proportion (% w/w) |
|---|---|
| Rosiglitazone (as maleate salt) | 1.1 |
| HPMC | 30.0 |
| Lactose | 66.9 |
| Silicon dioxide | 0.5 |
| Magnesium stearate | 1.5 |

by compression to form 7 mm normal concave bilayer tablets of 200 mg (50 mg of the immediate release layer and 150 mg of the modified release layer).

The tablet cores were coated with a HPMC-based sub-coat and a polymethacrylate resin soluble at pH 5.5 to a total weight of 217.3 mg.

An opening of diameter 3.0 mm was drilled through the coating in each of the two primary surfaces of the coated cores to expose the surface of the core.

The final tablet contained 2 mg rosiglitazone-0.75 mg rosiglitazone within the immediate release layer and 1.25 mg rosiglitazone within the modified release layer.

### (b) 4 mg Rosiglitazone Extended Release Tablet

A core was formed from the following compositions:

TABLE 4
4 mg rosiglitazone tablet first composition (immediate release layer).

| Component | Proportion (% w/w) |
|---|---|
| Rosiglitazone (as maleate salt) | 3.98 |
| Lactose | 95.49 |
| Yellow iron oxide | 0.03 |
| Magnesium stearate | 0.5 |

TABLE 5
4 mg rosiglitazone tablet second composition (modified release layer).

| Component | Proportion (% w/w) |
|---|---|
| Rosiglitazone (as maleate salt) | 2.2 |
| HPMC | 30.0 |
| Lactose | 65.8 |
| Silicon dioxide | 0.5 |
| Magnesium stearate | 1.5 |

by compression to form 7 mm normal concave bilayer tablets of 200 mg (50 mg of the immediate release layer and 150 mg of the modified release layer).

The tablet cores were coated with a HPMC-based sub-coat and a polymethacrylate resin soluble at PH 5.5 to a total weight of 217.3 mg.

An opening of diameter 3.0 mm was drilled through the coating in each of the two primary surfaces of the coated cores to expose the surface of the core.

The final tablet contained 4 mg rosiglitazone—1.5 mg rosiglitazone within the immediate release layer and 2.5 mg rosiglitazone within the modified release layer.

### (a) 8 mg Rosiglitazone Maleate Extended Release Tablet

A core was formed from the following compositions:

TABLE 6
8 mg rosiglitazone tablet first composition (immediate release layer).

| Component | Proportion (% w/w) |
|---|---|
| Rosiglitazone (as maleate salt) | 7.95 |
| Lactose | 91.52 |
| Yellow iron oxide | 0.03 |
| Magnesium stearate | 0.5 |

TABLE 7
8 mg rosiglitazone tablet second composition (modified release layer).

| Component | Proportion (% w/w) |
|---|---|
| Rosiglitazone (as maleate salt) | 4.4 |
| HPMC | 30.0 |
| Lactose | 63.6 |
| Silicon dioxide | 0.5 |
| Magnesium stearate | 1.5 |

by compression to form 7 mm normal concave bilayer tablets of 200 mg (50 mg of the immediate release layer and 150 mg of the modified release layer).

The tablet cores were coated with a HPMC-based sub-coat and a polymethacrylate resin soluble at PH 5.5 to a total weight of 217.3 mg.

An opening of diameter 3.0 mm was drilled through the coating in each of the two primary surfaces of the coated cores to expose the surface of the core.

The final tablet contained 8 mg rosiglitazone—3 mg rosiglitazone within the immediate release layer and 5 mg rosiglitazone within the modified release layer.

### Example 2

### The Effect of PPAR-Gamma Agonist (Rosiglitazone Maleate) Treatment on ADAS-Cog and CIBIC+ in Alzheimer's Patients

### Method

Analysis for the full intent to treat (ITT) population was performed on 511 subjects who were randomly allocated into one of four specific treatment regimes. Genotyping analysis was performed on 63% (323/511) of the ITT population.

Patient population included Caucasian males and females between 50-85 years of age who had been diagnosed with mild to moderate AD, were not receiving any medications which could adversely prejudice the study (e.g. PPAR-gamma agonists or conventional AD medicaments) or had any other potentially prejudicial ailments (e.g. diabetes or major psychiatric disorders).

Patients received either placebo or one of three dosage levels of extended release rosiglitazone provided once daily (2 mg, 4 mg and 8 mg tablets as described in Example 1). Patients were examined using the cognitive Alzheimer's Disease Assessment Scale (ADAS-cog; for further information see Rosen W G et al. Am. J. Psychiatry. 1984 141:1356-1364) and the Clinician's Interview-Based Impression of Change with caregiver information (CIBIC+; for further information see Knopman D S et al. Neurology 1994 44: 2315-2321); and secondary assessments were performed using: the Disability Assessment for Dementia (DAD, for further information see Gelinas L et al. Am J Occup Ther 1999 53: 471-81) and the Neuropsychiatric Inventory test (NPI, for further information see Cummings et al (1994) Neurology 44, 2308-2314) at the start of the study (baseline) and during the course of the study (after 8, 16 and 24 weeks of treatment).

APOE genotype was determined using the TaqMan PCR-based method of McLeod et al 2001 infra.

All statistics reflect last observed assessment carried forward (LOCF) measurements.

Tables 8 and 9 summarise the age and sex details of the genotyped and full ITT populations by treatment regime.

TABLE 8
Summary of genotyped population.

| | | | Rosiglitazone | Rosiglitazone | Rosiglitazone | |
|---|---|---|---|---|---|---|
| | | Placebo | 2 mg | 4 mg | 8 mg | Total |
| | | N = 78 | N = 85 | N = 80 | N = 80 | N = 323 |
| Age | Mean | 71.2 | 70 | 68.8 | 70.5 | 70.1 |
| | (SD) | (8.94) | (8.58) | (9.56) | (8.02) | (8.79) |
| Sex | Female | 51 | 53 | 47 | 54 | 205 |
| | | (65%) | (62%) | (59%) | (68%) | (63%) |
| | Male | 27 | 32 | 33 | 26 | 118 |
| | | (35%) | (38%) | (41%) | (33%) | (37%) |

TABLE 9
Summary of full intent to treat population.

| | | | Rosiglitazone | Rosiglitazone | Rosiglitazone | |
|---|---|---|---|---|---|---|
| | | Placebo | 2 mg | 4 mg | 8 mg | Total |
| | | N = 122 | N = 127 | N = 130 | N = 132 | N = 511 |
| Age | Mean | 71.8 | 70.9 | 69.7 | 70.5 | 70.7 |
| | (SD) | (8.23) | (8.46) | (8.97) | (8.47) | (8.55) |
| Sex | Female | 77 | 71 | 73 | 87 | 308 |
| | | (63%) | (56%) | (56%) | (66%) | (60%) |
| | Male | 45 | 56 | 57 | 45 | 203 |
| | | (37%) | (44%) | (44%) | (34%) | (40%) |

### Results

It should be noted that in the case of ADAS-cog higher scores indicate reduced cognitive function. A negative change from baseline over the course of the study therefore shows an improvement and a positive change from baseline shows decline. Similarly a negative treatment difference shows that treatment resulted in improvement relative to placebo and a positive treatment difference shows that treatment resulted in decline relative to placebo.

Higher CIBIC+ scores indicate a greater level of decline with scores below 4 denoting clinical improvement and scores above 4 denoting clinical decline. A negative CIBIC+ treatment difference therefore shows that treatment resulted in an improvement relative to placebo and a positive treatment difference shows that treatment resulted in decline relative to placebo.

### ITT Population

Table 10 summarises the model adjusted change in ADAS-cog from baseline and CIBIC+ results at the end of the 24 week trial for each of the four treatment regimes in the ITT population. FIG. 1 shows the model adjusted ADAS-cog change from baseline in the ITT population during the course of the study (the analyses included adjustments for effects of baseline score, country, mini mental state examination screening and baseline body mass index).

The ADAS-cog data in Table 10 and FIG. 1 support a trend of clinical improvement (i.e. a negative change from baseline) as a result of treatment using the PPAR-gamma agonist rosiglitazone. At all time points there is a net improvement in the analysed population as a whole. However statistical analysis of the effect of rosiglitazone treatment on AD patients indicates that this trend is not statistically significant. The CIBIC+ results did not lead to a distinguishable difference between treatment groups and placebo at 24 weeks.

TABLE 10
Summary of model adjusted ADAS-cog change from baseline after 24
weeks by treatment group (LOCF - ITT population).

| | | | | P-value |
|---|---|---|---|---|
| | | | Treatment Difference | for |
| | | | (95% confidence limits) | Treatment |
| Variable | Treatment Regime | LSMean (SE) | Rosi − Placebo | Difference |
| ADAS- | Placebo (n = 122) | −0.4 (0.55) | | |
| cog | Rosi 2 mg (n = 126) | −0.2 (0.54) | 0.25 (−1.19, 1.68) | 0.74 |
| | Rosi 4 mg (n = 128) | −0.9 (0.54) | −0.46 (−1.90, 0.97) | 0.52 |
| | Rosi 8 mg (n = 130) | −0.7 (0.53) | −0.27 (−1.70, 1.16) | 0.71 |
| CIBIC+ | Placebo (n = 122) | 4.0 (0.10) | | |
| | Rosi 2 mg (n = 126) | 3.8 (0.10) | −0.16 (−0.44, 0.11) | 0.23 |
| | Rosi 4 mg (n = 128) | 3.8 (0.10) | −0.16 (−0.43, 0.11) | 0.24 |
| | Rosi 8 mg (n = 130) | 3.8 (0.10) | −0.22 (−0.49, 0.05) | 0.11 |

### Genotyped Population

Table 11 and Table 11 a (which reflects the inclusion of 2 additional subjects) indicate the results of APOE4 allele determination in the genotypes population. Treatment regimes were allocated prior to APOE4 allele determination, despite this, there is generally a good distribution of phenotypes between the various groupings as a result of statistical averaging, although some of the less prevalent phenotypes show some clustering (for example a large proportion of the APOE4 homozygotes are in the 8 mg rosiglitazone treatment group).

TABLE 11
Summary of APOE allele status by treatment group.

| | | | | Rosi | Rosi | |
|---|---|---|---|---|---|---|
| | | | Rosi 2 mg | 4 mg | 8 mg | Total |
| | | Placebo N = 78 | N = 85 | N = 80 | N = 80 | N = 323 |
| APOE | N | 78 (100%) | 85 (100%) | 79 (100%) | 78 (100%) | 320 (100%) |
| Genotype | 4, 4 | 5 (6%) | 4 (5%) | 6 (8%) | 12 (15%) | 27 (8%) |
| | 3, 4 | 27 (35%) | 31 (37%) | 27 (34%) | 22 (28%) | 107 (33%) |
| | 2, 4 | 3 (4%) | 1 (1%) | 1 (1%) | 2 (3%) | 7 (2%) |
| | 3, 3 | 35 (45%) | 43 (51%) | 37 (47%) | 35 (45%) | 150 (47%) |
| | 2, 3 | 8 (10%) | 6 (7%) | 7 (9%) | 7 (9%) | 28 (9%) |
| | 2, 2 | 0 | 0 | 1 (1%) | 0 | 1 (<1%) |
| APOE4 | 2 | 5 (6%) | 4 (5%) | 6 (8%) | 12 (15%) | 27 (8%) |
| Copies | 1 | 30 (38%) | 32 (38%) | 28 (35%) | 24 (31%) | 114 (36%) |
| | 0 | 43 (55%) | 49 (58%) | 45 (57%) | 42 (54%) | 179 (56%) |
| APOE4 | Yes | 35 (45%) | 36 (42%) | 34 (43%) | 36 (46%) | 141 (44%) |
| Carriage | No | 43 (55%) | 49 (58%) | 45 (57%) | 42 (54%) | 179 (56%) |

TABLE 11a
Summary of APOE allele status by treatment group.

| | | | Rosi | Rosi | Rosi | |
|---|---|---|---|---|---|---|
| | | | 2 mg | 4 mg | 8 mg | Total |
| | | Placebo N = 78 | N = 85 | N = 80 | N = 80 | N = 323 |
| APOE | N | 78 (100%) | 85 (100%) | 80 (100%) | 79* (100%) | 322 (100%) |
| Genotype | 4, 4 | 5 (6%) | 4 (5%) | 6 (8%) | 12 (15%) | 27 (8%) |
| | 3, 4 | 27 (35%) | 31 (37%) | 28 (35%) | 22 (28%) | 108 (34%) |
| | 2, 4 | 3 (4%) | 1 (1%) | 1 (1%) | 2 (3%) | 7 (2%) |
| | 3, 3 | 35 (45%) | 43 (51%) | 37 (46%) | 36 (46%) | 151 (47%) |
| | 2, 3 | 8 (10%) | 6 (7%) | 7 (9%) | 7 (9%) | V28 (9%) |
| | 2, 2 | 0 | 0 | 1 (1%) | 0 | 1 (<1%) |
| APOE4 | 2 | 5 (6%) | 4 (5%) | 6 (8%) | 12 (15%) | 27 (8%) |
| Copies | 1 | 30 (38%) | 32 (38%) | 29 (36%) | 24 (30%) | 115 (36%) |
| | 0 | 43 (55%) | 49 (58%) | 45 (56%) | 43 (54%) | 180 (56%) |
| APOE4 | Yes | 35 (45%) | 36 (42%) | 35 (44%) | 36 (46%) | 142 (44%) |
| Carriage | No | 43 (55%) | 49 (58%) | 45 (56%) | 43 (54%) | 180 (56%) |

*no genotype information available for one subject

A breakdown of the change in ADAS-cog at the end of the 24 week study by APOE allele status and treatment regime is shown in Table 12 below.

A prospectively defined test for interaction between APOE carriage status and ADAS-cog total score change from baseline to week 24 was significant (P=0.0194). Subsequent exploratory testing revealed that APOE4− (those without an APOE4 allele) patients, after 24 weeks, showed a general trend of improvement in cognitive function as a result of treatment with the PPAR-gamma agonist rosiglitazone, there being evidence that this improvement was due to treatment at the highest 8 mg rosiglitazone dosage compared to placebo (P=0.027).

APOE4 heterozygotes (those with a single APOE4 allele) do not show any recognisable trend. Although there is some decline in the group receiving 2 mg rosiglitazone, both the 4 mg and 8 mg dose regimes show little change, and none of the points are individually significant after 24 weeks of treatment.

APOE4 homozygotes (those with two APOE4 alleles) show a relatively large positive change in ADAS-cog scores as a result of rosiglitazone treatment. There was some evidence that this decline was due to treatment at all three dosage levels after 24 weeks of treatment (unadjusted P<0.05), although sample numbers are small. However, the extent of clinical decline as a result of treatment decreases with increasing dosage level. It is not clear whether the clinical decline in the treated group is due to rosiglitazone or due to the natural progression of Alzheimer's disease.

TABLE 12
Summary of model-adjusted ADAS-cog change from baseline after 24
weeks by APOE4 allele status and treatment group (PGx ITT population).

| | | | Treatment Difference | P-value for |
|---|---|---|---|---|
| APOE4 | Treatment | | (90% confidence limits) | Treatment |
| Copies | Regime | LSMean (SE) | Rosi − Placebo | Difference |
| 0 | Placebo (n = 43) | 1.01 (0.95) | | |
| | Rosi 2 mg (n = 49) | −1.38 (0.90) | −2.39 (−4.43, −0.36) | 0.053 |
| | Rosi 4 mg (n = 45) | −1.25 (0.90) | −2.26 (−4.32, −0.20) | 0.071 |
| | Rosi 8 mg (n = 42) | −1.85 (0.94) | −2.86 (−4.98, −0.74) | 0.027 |
| 1 | Placebo (n = 30) | −0.57 (1.10) | | |
| | Rosi 2 mg (n = 32) | 2.02 (1.10) | 2.59 (0.10, 5.08) | 0.087 |
| | Rosi 4 mg (n = 28) | −0.21 (1.15) | 0.36 (−2.18, 2.90) | 0.82 |
| | Rosi 8 mg (n = 24) | −0.34 (1.25) | 0.23 (−2.42, 2.87) | 0.89 |
| 2 | Placebo (n = 5) | −4.58 (2.70) | | |
| | Rosi 2 mg (n = 4) | 5.67 (2.98) | 10.26 (3.64, 16.87) | 0.011 |
| | Rosi 4 mg (n = 6) | 3.16 (2.44) | 7.75 (1.79, 13.71) | 0.033 |
| | Rosi 8 mg (n = 12) | 1.91 (1.73) | 6.50 (1.28, 11.71) | 0.041 |

FIG. 2 shows a plot of the model adjusted ADAS-cog change from baseline in the analysed population by treatment regime and APOE allele status (carriers of 1 or 2 APOE4 alleles being shown together). FIG. 3 shows a plot in which data on the APOE4 heterozygotes (indicated by ‘Het E4+’) have been separated from data on the APOE4 homozygotes (indicated by ‘Homo E4+’).

A clear trend of cognitive improvement as a result of rosiglitazone treatment is particularly apparent in the APOE4− individuals. At all time points (8, 16 and 24 weeks) the placebo group shows a continued decline in cognitive function, whereas those treated with 2 mg, 4 mg or 8 mg of the PPAR-gamma agonist show marked improvement.

The situation with respect to APOE4+ individuals is less clear. After 8 weeks of treatment, those receiving placebo show a slight decline in cognitive function, while all those receiving rosiglitazone (2 mg, 4 mg or 8 mg) slow slight improvement.

After 16 weeks of treatment those receiving placebo show a continued decline in cognitive function, although treatment with 4 mg and 8 mg shows the same or better clinical status. Treatment with 2 mg rosiglitazone shows a greater decline than the placebo. Finally, after 24 weeks of treatment, a large and surprising improvement in APOE4 carriers receiving placebo is observed. This apparent improvement may have been influenced by a small number of subjects with unexpected and large improvements in ADAS-cog scores. All three rosiglitazone treatment arms finish with a clinical decline, and as a result of the unusual improvement in the placebo arm at this time point, rosiglitazone treatment appears to depict a clinical decline compared to the placebo. It is possible that the clinical decline observed in some APOE4+ groups is due to the natural clinical course of AD.

FIG. 3 shows the results for the APOE4 heterozygotes separated from those for the APOE4 homozygotes. Although the number of APOE4 homozygotes is small, it can be seen that whereas all APOE4 homozygotes treated with rosiglitazone experienced a clinical decline, the APOE4 heterozygotes who received the higher doses of rosiglitazone (4, 8 mg) remained close to the baseline for the course of the study.

Similar results were identified using the Disability Assessment for Dementia (DAD) test (Gelinas L et al. Am J Occup Ther 1999 53: 471-81). A prospectively defined test for interaction between APOE4 carriage status and DAD scores at week 24 was significant (P=0.006). Subsequent testing demonstrated a pattern of results that is qualitatively similar to that for ADAS-Cog: namely, APOE4− subjects demonstrated improvement on DAD, whereas APOE4+ subjects demonstrated no improvement.

Similar results were identified using the Neuropsychiatric Inventory (NPI) test (Cummings et al (1994) Neurology 44, 2308-2314). A prospectively defined test for interaction between APOE4 carriage status and NPI scores at week 24 was significant (P=0.086). Subsequent testing demonstrated a pattern of results that is qualitatively similar to that for ADAS-Cog: namely, APOE4− subjects demonstrated improvement on NPI, whereas APOE4+ subjects demonstrated no improvement.

Table 13 and Table 13a (which is an updated analysis taking into account additional subjects) shows the CIBIC+ results after 24 weeks, separated by APOE4 allele status and treatment regime. There was no evidence of an interaction between treatment and APOE4 copies, so the differences described below between the subgroups are likely to be due to random error rather than any differential effect.

APOE4− (those without an APOE4 allele) patients all show slight improvement over the 24 week period, with the greatest improvement observed in the group treated with 2 mg of rosiglitazone (unadjusted P=0.052).

APOE4 heterozygotes (those with a single APOE4 allele) show a decline in the group treated with 2 mg of rosiglitazone (P=0.056). Less decline is shown in the group receiving 4 mg of rosiglitazone and a slight improvement is seen in the group receiving 8 mg rosiglitazone (although no comparison came close to significance in the exploratory analysis).

APOE4 homozygotes (those with two APOE4 alleles) all slow slight improvement in the CIBIC+ upon treatment for 24 weeks compared to placebo, although the extent of improvement decreased with treatment dosage.

TABLE 13
Summary of model adjusted CIBIC+ after 24 weeks by APOE4 allele
status and treatment group (PGx ITT population).

| | | | Treatment Difference | P-value for |
|---|---|---|---|---|
| APOE4 | Treatment | | (90% confidence limits) | Treatment |
| Copies | Regime | LSMean (SE) | Rosi − Placebo | Difference |
| 0 | Placebo (n = 41) | 3.95 (0.19) | | |
| | Rosi 2 mg (n = 45) | 3.47 (0.18) | −0.49 (−0.89, −0.08) | 0.051 |
| | Rosi 4 mg (n = 43) | 3.76 (0.18) | −0.19 (−0.60, 0.22) | 0.44 |
| | Rosi 8 mg (n = 42) | 3.76 (0.18) | −0.19 (−0.61, 0.22) | 0.44 |
| 1 | Placebo (n = 28) | 3.88 (0.22) | | |
| | Rosi 2 mg (n = 28) | 4.47 (0.23) | 0.59 (0.08, 1.11) | 0.056 |
| | Rosi 4 mg (n = 25) | 4.11 (0.23) | 0.23 (−0.28, 0.74) | 0.45 |
| | Rosi 8 mg (n = 23) | 3.68 (0.25) | −0.20 (−0.73, 0.34) | 0.54 |
| 2 | Placebo (n = 5) | 4.34 (0.53) | | |
| | Rosi 2 mg (n = 4) | 3.42 (0.58) | −0.92 (−2.20, 0.37) | 0.24 |
| | Rosi 4 mg (n = 6) | 3.95 (0.47) | −0.39 (−1.55, 0.77) | 0.58 |
| | Rosi 8 mg (n = 12) | 4.27 (0.34) | −0.07 (−1.08, 0.95) | 0.91 |

TABLE 13A
Summary of model adjusted CIBIC+ after 24 weeks by APOE4 allele
status and treatment group (PGx ITT population).

| | | | Treatment Difference | P-value for |
|---|---|---|---|---|
| APOE4 | Treatment | | (90% confidence limits) | Treatment |
| Copies | Regime | LSMean (SE) | Rosi − Placebo | Difference |
| 0 | Placebo (n = 43) | 3.97 (0.18) | | |
| | Rosi 2 mg (n = 49) | 3.51 (0.17) | −0.46 (−0.85, −0.07) | 0.052 |
| | Rosi 4 mg (n = 44) | 3.75 (0.17) | −0.22 (−0.62, 0.18) | 0.37 |
| | Rosi 8 mg (n = 43) | 3.75 (0.18) | −0.22 (−0.62, 0.19) | 0.38 |
| 1 | Placebo (n = 30) | 3.92 (0.21) | | |
| | Rosi 2 mg (n = 31) | 4.32 (0.21) | 0.39 (−0.09, 0.87) | 0.18 |
| | Rosi 4 mg (n = 29) | 3.97 (0.22) | 0.04 (−0.44, 0.53) | 0.88 |
| | Rosi 8 mg (n = 23) | 3.70 (0.24) | −0.22 (−0.74, 0.29) | 0.48 |
| 2 | Placebo (n = 5) | 4.38 (0.52) | | |
| | Rosi 2 mg (n = 4) | 3.46 (0.57) | −0.91 (−2.19, 0.36) | 0.24 |
| | Rosi 4 mg (n = 6) | 3.93 (0.47) | −0.45 (−1.59, 0.70) | 0.52 |
| | Rosi 8 mg (n = 12) | 4.29 (0.33) | −0.09 (−1.09, 0.92) | 0.89 |

APOE4 copies *treatment interaction P-value = 0.21

### Discussion

The results of Example 2 show that treatment of AD patients using the PPAR-gamma agonist rosiglitazone leads to a non-statistically significant trend to a general improvement in the ITT population as a whole.

In the population tested, there was evidence of a cognitive improvement (as measured in ADAS-cog) in patients without the APOE4 allele on 8 mg rosiglitazone. The mean change from placebo over 24 weeks in patients without the APOE4 allele on 8 mg rosiglitazone was −2.86, P=0.027.

In the population tested, there was no evidence of on-treatment cognitive improvement (as measured by ADAS-cog) in patients carrying the APOE4 allele. However separation of patients carrying one copy from those carrying two copies of the APOE4 allele suggests greatest cognitive decline (as measured by ADAS-cog) in patients carrying two copies of the APOE4 allele (which may, however, be due to the natural progression of the disease rather than response to rosiglitazone) with no notable trend (eg possible stabilisation of cognitive function) in patients carrying one copy of the APOE4 allele.

### Example 3

### Oral Dosage Forms Containing Rosiglitazone (Eg as Maleate) And Donepezil (Eg as Hydrochloride)

### Formulation A

Formulation A has a bilayer tablet core comprising an immediate release layer and a modified release layer as shown in Table 14. This tablet is then coated and drilled to form a DiffCORE tablet (in a manner similar to that described in WO2005/013935).

TABLE 14
Composition of donepezil/rosiglitazone DiffCORE Formulae

| Component | Proportion (% w/w) |
|---|---|
| Immediate Release Layer | |
| Rosiglitazone (pfb) | 1 to 10¹ |
| Donepezil (pfb) | 1 to 10² |
| Magnesium Stearate | 0.5 |
| Yellow Iron Oxide | 0.03 |
| Lactose Monohydrate | to 100 |
| Modified-Release Layer | |
| Rosiglitazone (pfb) | 1 to 10¹ |
| Hypromellose 2208 | 30.0 |
| Silicon Dioxide | 0.5 |
| Magnesium Stearate | 1.5 |
| Lactose Monohydrate | to 100 |

NOTES:
¹rosiglitazone quantity as 2 mg, 4 mg or 8 mg per dose (rosiglitazone may suitably be used as rosiglitazone maleate)
NOTES:
²donepezil quantity as 5 mg or 10 mg per dose (donepezil may suitably be used as donepezil hydrochloride)

The tablet cores were coated with a HPMC-based sub-coat and a polymethacrylate resin soluble at pH 5.5.

An opening of diameter 3.0 mm was drilled through the coating in each of the two primary surfaces of the coated cores to expose the surface of the core.

The final tablet contained 2 mg, 4 mg or 8 mg rosiglitazone (0.75 mg, 1.5 mg or 3 mg rosiglitazone within the immediate release layer and 1.25 mg, 2.5 mg or 5 mg rosiglitazone within the modified release layer respectively) along with either 5 mg or 10 mg donepezil in the immediate release layer.

### Formulation B

Formulation B is an enteric matrix controlled bilayer tablet.

In Table 15 below the donepezil has been incorporated into the immediate release layer of the tablet for the range of possible strength combinations:

TABLE 15
Composition of donepezil/rosiglitazone Enteric Matrix Tablet Formulae

| Component | Proportion (% w/w) |
|---|---|
| Immediate Release | |
| Layer | |
| Rosiglitazone (pfb) | 1 to 10¹ |
| Donepezil (pfb) | 1 to 10² |
| Magnesium Stearate | 0.5 |
| Yellow Iron Oxide | 0.03 |
| Lactose Monohydrate | to 100 |
| Modified-Release | |
| Layer | |
| Rosiglitazone (pfb) | 1 to 10¹ |
| Enteric Polymer | 40.0 |
| Polyethylene Glycol | 18.0 |
| Magnesium Stearate | 1.0 |
| Dicalcium Phosphate | To 100 |

NOTES:
¹Rosiglitazone quantity as 2 mg, 4 mg or 8 mg per dose (rosiglitazone may suitably be used as rosiglitazone maleate
NOTES:
²Donepezil quantity as 5 mg or 10 mg per dose (donepezil may suitably be used as donepezil hydrochloride)

The final tablet contained 2 mg, 4 mg or 8 mg rosiglitazone (0.75 mg, 1.5 mg or 3 mg rosiglitazone within the immediate release layer and 1.25 mg, 2.5 mg or 5 mg rosiglitazone within the modified release layer respectively) along with either 5 mg or 10 mg donepezil in the immediate release layer.

### Formulation C

Formulation C comprises drug coated pellets filled into capsules. The capsules are filled with a mixture comprised of immediate release rosiglitazone, immediate release donepezil and enteric coated rosiglitazone pellets. The pellet formulation is shown in Table 16:

TABLE 16
Composition of donepezil/rosiglitazone pellet formulation for capsules

| | |
|---|---|
| Component | Proportion (% w/w) |

| | | |
|---|---|---|
| Immediate Release | Rosiglitazone | Donepezil |
| Pellets | IR | IR |
| Rosiglitazone (pfb) | 1 to 10¹ | — |
| Donepezil (pfb) | — | 1 to 10² |
| Polysorbate 80 | 1.0 | 1.0 |
| Clear Hypromellose Based | 3.0 | 3.0 |
| Coat¹ | | |
| Sugar Spheres (25/30 | To 100 | To 100 |
| mesh) | | |

NOTES:
¹Rosiglitazone quantity as 2 mg, 4 mg or 8 mg per dose (rosiglitazone may suitably be used as rosiglitazone maleate)
NOTES:
²Donepezil quantity as 5 mg or 10 mg per dose (donepezil may suitably be used as donepezil hydrochloride)

The rosiglitazone and donepezil immediate release pellets were coated with a HPMC-based sub-coat. A portion of the sub-coated pellets were enteric coated with a polymethacrylate resin soluble at pH 5.5.

The final capsule contained 2 mg, 4 mg or 8 mg rosiglitazone (0.75 mg, 1.5 mg or 3 mg rosiglitazone in immediate release pellets and 1.25 mg, 2.5 mg or 5 mg rosiglitazone within the modified release pellets respectively) along with either 5 mg or 10 mg donepezil in immediate release pellets.

All references referred to in this application, including patents and patent applications, are incorporated herein by reference to the fullest extent possible. Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

1. A composition comprising rosiglitazone or a pharmaceutically acceptable salt thereof and donepezil or a pharmaceutically acceptable salt thereof together with a pharmaceutically acceptable diluent or carrier.

2. (canceled)

3. A method for improving cognitive function in a subject suffering from or susceptible to mild cognitive impairment, Alzheimer's disease or other dementias, the method comprising administering to the subject a safe and effective amount of a composition comprising rosiglitazone or a pharmaceutically acceptable salt thereof and donepezil or a pharmaceutically acceptable salt thereof.

4-5. (canceled)

6. A method as claimed in claim 3, wherein the subject is not homozygous for the APOE4 allele.

7. A method as claimed in claim 3, wherein the subject does not carry the APOE4 allele.

8. A method for improving cognitive function in a subject suffering from or susceptible to MCI, Alzheimer's disease or other dementias, which subject is not homozygous for the APOE4 allele, comprising the steps of:
i) screening the subject to determine that the subject is not homozygous for the APOE4 allele; and then
ii) administering a safe and effective amount of a composition comprising rosiglitazone or a pharmaceutically acceptable salt thereof and donepezil or a pharmaceutically acceptable salt thereof to said subject.

9. A method as claimed in claim 8, wherein the screening step (i) involves determining that the subject carries a single copy of the APOE4 allele.

10. A method as claimed in claim 8, wherein the screening step (i) involves determining that the subject is APOE4−.

11. A method as claimed in claim 8, wherein the subject is suffering from or susceptible to MCI (particularly amnestic MCI).

12. A method as claimed in claim 8, wherein the subject is suffering from or susceptible to Alzheimer's disease.

13. A method of screening a subject suffering from or susceptible to or susceptible to MCI, Alzheimer's disease or other dementias as an aid in predicting the subject's response to administration of a composition comprising rosiglitazone or a pharmaceutically acceptable salt thereof and donepezil or a pharmaceutically acceptable salt thereof, comprising screening to determine whether the subject carries zero or 1 copy of the APOE4 allele.

14. A method as claimed in claim 13 which includes screening to determine whether the subject is APOE4−.

15. A kit comprising (i) a composition comprising rosiglitazone or a pharmaceutically acceptable salt thereof and donepezil or a pharmaceutically acceptable salt thereof and (ii) instructions directing administration of the PPAR gamma agonist (typically in the form of a pharmaceutical composition) to a subject who is not homozygous for the APOE4 allele.

16. A composition as claimed in claim 1, wherein the rosiglitazone is in the form of rosiglitazone maleate.

17. A composition as claimed in claim 1, wherein the donepezil is in the form of donepezil hydrochloride.

18. A composition as claimed in claim 1 which is formulated for oral, parenteral, inhalation, transdermal, rectal and topical administration.

19. An oral dosage form comprising:
a first composition comprising rosiglitazone or a pharmaceutically acceptable salt thereof and donepezil or a pharmaceutically acceptable salt thereof formulated for immediate release on contact with aqueous media; and
a second composition comprising rosiglitazone or a pharmaceutically acceptable salt thereof formulated for modified release on contact with aqueous media.

20. An oral dosage form as claimed in claim 19, wherein the first and second compositions are multiparticulates.

21. An oral dosage form as claimed in claim 20 wherein the multiparticulates are drug-coated or drug-containing lactose spheres.

22. An oral dosage form as claimed in claim 20 the multiparticulates comprising the first composition are uncoated and the multiparticulates comprising the second composition have an enteric coat.

23. An oral dosage form as claimed in claim 20, wherein the multiparticulates are filled into a capsule.

24. An oral dosage form as claimed in claim 19 which takes the form of a tablet in which the first and second compositions are arranged in two or more separate layers.

25. An oral dosage form as claimed in claim 24 wherein the first and second compositions are arranged in two separate layers.

26. An oral dosage form as claimed in claim 24 wherein the immediate release composition includes a saccharose

27. An oral dosage form as claimed in claim 26, wherein the immediate release composition includes lactose and optionally magnesium stearate.

28. An oral dosage form as claimed in claim 19, wherein the modified release comprises delayed and/or sustained release.

29. An oral dosage form as claimed in claim 24, wherein the second composition comprises a sustained release composition in tablet formulation.

30. An oral dosage form as claimed in claim 20, wherein the second composition comprises a sustained release composition in the form of multiparticles coated with a semipermeable membrane.

31. An oral dosage formulation as claimed in claim 19 which is coated with an enteric coat.

32. An oral dosage formulation comprising an erodable core which comprises an oral dosage formulation as claimed in claim 19 and an erodable coating around the core, wherein the erodable coating comprises one or more openings extending substantially completely through said coating but not penetrating the core and communicating from the environment of use to the core, wherein release of the rosiglitazone or a pharmaceutically acceptable salt thereof and donepezil or a pharmaceutically acceptable salt thereof from the core occurs substantially through said opening(s) and through erosion of the coating under pre-determined pH conditions.

33. An oral dosage formulation as claimed in claim 32, wherein the erosion of the coating is pH dependent.

34. An oral dosage form as claimed in claim 33, wherein the erodable coating is an enteric coating.

35. An oral dosage form as claimed in claim 32 wherein the openings in the coat are in the range of about 0.19 to about 50.3 mm², which corresponds to a circular opening having a diameter in the range of 0.5 mm to 8 mm.

36. An oral dosage form as claimed in claim 32, wherein the core comprises discrete layers of the first and second compositions and the coat has two openings arranged such that a first opening provides access to the first composition and a second opening provides access to the second composition.

37. (canceled)

38. A method for improving cognitive function in a subject suffering from or susceptible to mild cognitive impairment, Alzheimer's disease or other dementias, the method comprising administering to the subject an oral dosage form as claimed in claim 19.

39. A method as claimed in claim 38, wherein the subject is not homozygous for the APOE4 allele.

40. A method as claimed in claim 38, wherein the subject does not carry the APOE4 allele.

41. A method for improving cognitive function in a subject suffering from or susceptible to MCI, Alzheimer's disease or other dementias, which subject is not homozygous for the APOE4 allele, comprising the steps of:
i) screening the subject to determine that the subject is not homozygous for the APOE4 allele; and then
ii) administering an oral dosage form as claimed in claim 19 to said subject.

42. A method as claimed in claim 41, wherein the screening step (i) involves determining that the subject carries a single copy of the APOE4 allele.

43. A method as claimed in claim 41, wherein the screening step (i) involves determining that the subject is APOE4−.

44. A method as claimed in claim 38, wherein the subject is suffering from or susceptible to MCI.

45. A method as claimed in claim 38, wherein the subject is suffering from or susceptible to Alzheimer's disease.

46. An oral dosage form as claimed in claim 19, wherein the rosiglitazone is in the form of rosiglitazone maleate.

47. An oral dosage form as claimed in claim 19, wherein the donepezil is in the form of donepezil hydrochloride.
